(19) United States
(12) Patent Application Publication
(10) Pub. No.: US 2016/0377628 A1
(43) Pub. Date: Dec. 29, 2016

(54) METHOD FOR QUANTITATIVE DETERMINATION OF MICRO MOLAR CONCENTRATION LEVEL OF D-GLUCOSE USING SURFACE ENHANCED RAMAN SPECTROSCOPY (SERS) WITH 2-THIENYLBORONIC ACID AS LINKER MOLECULE ON SILVER NANO-CLUSTER SUBSTRATES

(71) Applicants: Chandrahas Bansal, Hyderabad (IN); Ammanabrolu Rajanikanth, Hyderabad (IN); Raju Botta, Chandur Mandal (IN)

(72) Inventors: Chandrahas Bansal, Hyderabad (IN); Ammanabrolu Rajanikanth, Hyderabad (IN); Raju Botta, Chandur Mandal (IN)

(21) Appl. No.: 15/190,418

(22) Filed: Jun. 23, 2016

(30) Foreign Application Priority Data
Jun. 23, 2015 (IN) 3146/CHE/2015

(51) Int. Cl. G01N 33/66; G01N 21/65

(52) U.S. Cl. CPC G01N 33/66; G01N 21/658; G01N 2201/06113

(57) ABSTRACT

A method of determining micro-molar concentration of glucose in a body fluid using Surface Enhanced Raman Spectroscopy (SERS), comprising an analyte solution prepared by mixing body fluid and linker molecule solution, allowing the analyte solution to dry on the detection film of the glass substrate; recording and analyzing the glucose specific strong SERS signal in the Raman Spectra; and determining the presence of glucose at micro-molar level by measuring the strong SERS signal.

### RELATED APPLICATION

This application claims the benefit of priority of our Indian patent application number 3146CHE/2015 filed on Jun. 23, 2015, the content of which is hereby incorporated by reference herein in its entirety.

### TECHNICAL FIELD

The present subject matter in general relates to non-invasive method to determine micro molar concentration of glucose. More particularly the invention discusses the use of 2-thienylboronic acid as linker molecule for determining the level of D-Glucose using surface enhanced Raman spectroscopy.

### BACKGROUND

Diabetes is a metabolic disease which is one of the most leading causes for death in the world, affecting millions of people worldwide and resulting in long-term health disorders mainly cardiovascular diseases and blindness According to the IDF Diabetes Atlas published by the International Diabetes Federation in 2013, currently over 380 Million people are affected with Diabetes across the world, caused around 5.1 million deaths in 2013 i.e. every six seconds a person dies from diabetes. More than 21 million live births were affected by diabetes during pregnancy in 2013. In India, around 68 Million people are affected with Diabetes. It is also estimated that people living with diabetes will increase to 55% by 2035. So a simple and continuous monitoring method for glucose is very essential to address the disease.

The detection of glucose levels is important for the detection of diabetes. Various Glucose detection methods especially dry Chemistry test methods are currently available in the market. Non-invasive detection method is very preferred method where the samples like urine or Saliva are collected for glucose examination, apart from the invasive detection method where the samples like blood are collected for glucose examination.

Surface Enhanced Raman Spectroscopy (SERS) is one spectroscopic technique which uses Raman scattering for enhancing the detection of molecular species through the excitation of Plasmon modes and their coupling to molecular vibrational modes. The substrate surface on which the detection of molecular species is taking place, as well as the material of the substrate surface affects the strength and intensity of the Raman scattering. This technique is considered to be a highly surface-sensitive enough to detect single molecule which enhances the Raman scattering by 10⁴-10⁶ times through molecules adsorbed on rough metal surfaces, for example: Au, Ag, Cu and Pt.

The use of Surface Enhanced Raman Spectroscopy (SERS) has been studied to detect the level of glucose by examining the samples at the molecular level. U.S.Dinish et al. in his publication in Biosensors & Bioelectronics, Volume 26, Issue 5, 15 Jan. 2011, mentioned that Detection of glucose by Surface Enhanced Raman Spectroscopy (SERS) is limited by two factors. One factor is the low Raman scattering cross-section of the glucose molecule; and the second factor is the poor affinity of glucose molecules to be adsorbed on metal surface which has been highlighted by Shafer-Peltier et al. in Journal of the American Chemical Society, 2003 Jan. 15 and Jonathan et al. in Analytical Chemistry, 2010 Oct. 15.

Many approaches have been studied and taken to address the aforementioned challenges. Shafer-Peltier et al. came up with an idea to modify the metal surface chemically with alkane thiol group. In this method, alkane thiol is self-assembled on a silver film over nano-sphere to act as a partition layer to concentrate D-glucose molecules near the surface of silver. This method measures the Surface Enhanced Raman Spectroscopy signals from the D-glucose concentrated near the Silver surface, however the D-glucose Raman signal is very weak and was not used to quantify D-glucose concentration. In a similar work carbohydrate recognition molecules such as boronic acid and phenyl boronic acid were used to increase the concentration of captured D-glucose. A mixed decanethiol (DT)/mercaptohexanol (MH) partition layer with dramatically improved properties is also investigated in vivo by K. Ma et al. and was published in Analytical Chemistry, 2011 Dec. 1.

Some other surface enhanced Raman spectroscopy substrates have also been studied by Kanayama et al, Langmuir (2000); Barriet D et al, Langmuir (2007); where the thiol containing molecule was used to attach the molecule to the metal surface but the trapping near the metal surface was not specific to glucose.

In another study published in Biosensor and Bioelectronics; 2014 Jun. 15, Kong et al. proposed a mechanism in order to overcome challenges in specific glucose detection in bio-fluids. They used phenylboronic acid as a receptor for saccharide capture onto the substrate and allowed captured glucose molecule to undergo secondary binding with an alkyne-functionalized boronic acid to form a glucose-alkyne-boronic acid complex. This complex was used as Surface Enhanced Raman Spectroscopy active substrates for detection of glucose. The alkyne group exhibited Raman signal intensity at 1996 cm⁻¹ and was shown to change with glucose concentration. The Raman signals from the alkyne radicals were measured but were very weak in intensity and measurable only up to 0.5 milli molar concentration levels, also this is an indirect method of measuring the glucose concentration.

With this background, there is a need for an effective method which can be helpful in selective adsorption of glucose molecule to enhance the Raman Signal intensity and hence quantify glucose concentration at micro molar level.

Thus, in the view of above mentioned reasons, the present invention discusses a direct and cost effective method for determining the micro molar concentration of glucose in a body fluid.

### OBJECT OF THE INVENTION

The principal object of the invention is to provide a method for quantitative determination of micro molar concentration of D-glucose using Surface Enhanced Raman Spectroscopy.

Another object of this invention is to provide a detection film on a glass substrate by physical vapor deposition technique having no other chemical residue.

Yet another object of this invention is to provide a method of use of linker molecule which attaches selectively with glucose molecules present in the body fluid and detection film present on the glass substrate and hence provide a strong SERS signal; which can be helpful in determining the presence of glucose in the body fluid micro molar level.

### SUMMARY OF THE INVENTION

The present invention relates to a method of determining micro-molar concentration of glucose in a body fluid using Surface Enhanced Raman Spectroscopy (SERS), the method comprising
a. preparing a solution by mixing body fluid whose glucose concentration has to be determined and pre-determined concentration of linker molecule solution capable of selectively attaching to the glucose molecule,
b. dropping the said solution on surface composed of
   i.a glass substrate;
   ii.a detection film deposited on the surface of the glass substrate capable of binding the linker molecule;
c. permitting the said surface to dry naturally under ambient conditions;
d. recording and analyzing the glucose specific strong SERS signal in the Raman Spectra; and
e. determining the presence of glucose at micro-molar level by measuring the strong SERS signal.

In another aspect, the present invention provides a fabrication of detection film using physical vapor deposition technique and depositing it on a glass substrate in such a way that it is free from any other chemical residue.

In another aspect, the present invention provides a method of using a linker molecule that has a unique property of selectively attaching to the glucose molecule present in body fluid and further gets linked to the detection film present on the glass substrate. This linker molecule has strong affinity for both the glucose molecule and detection film which helps in getting the strong SERS signal in the Raman spectra.

In another aspect, the present invention provides to a direct and non-invasive method for determining the presence of the glucose in the body fluid.

BRIEF DESCRIPTION OF THE ACCOMPANYING FIGURES

The above and other aspects, features, and advantages of certain exemplary embodiments of the present invention will be more apparent from the following description taken in conjunction with the accompanying drawings in which:

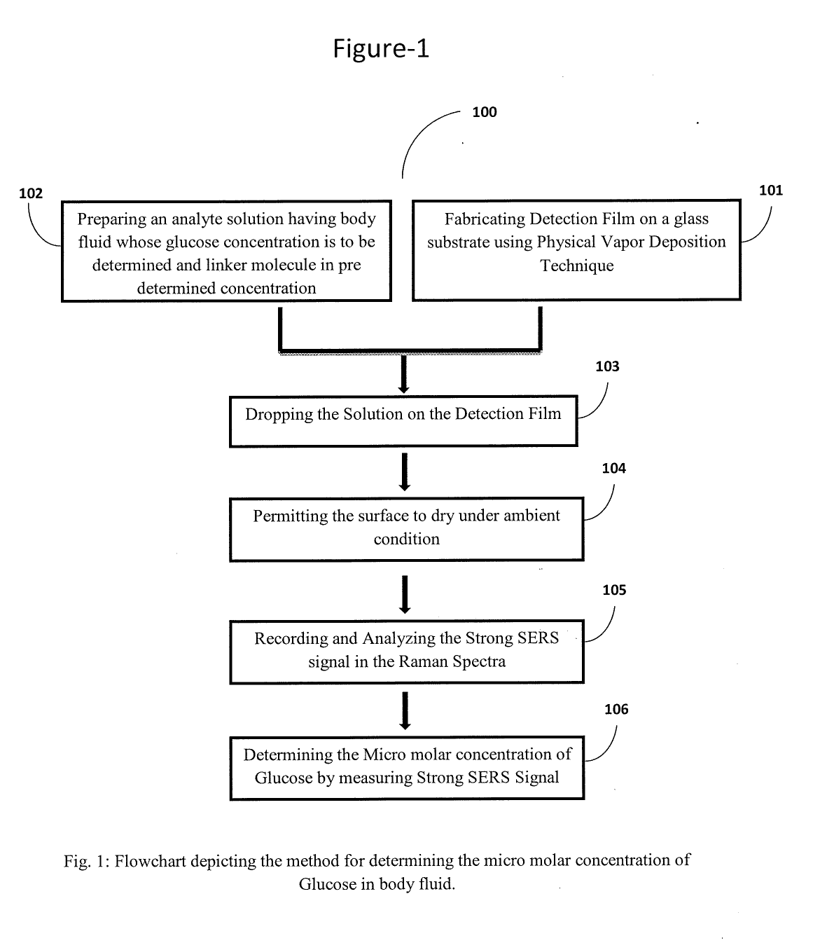

FIG. 1 shows flowchart depicting the method for determining the micro molar concentration of Glucose in body fluid using the Surface Enhanced Raman Spectroscopy.

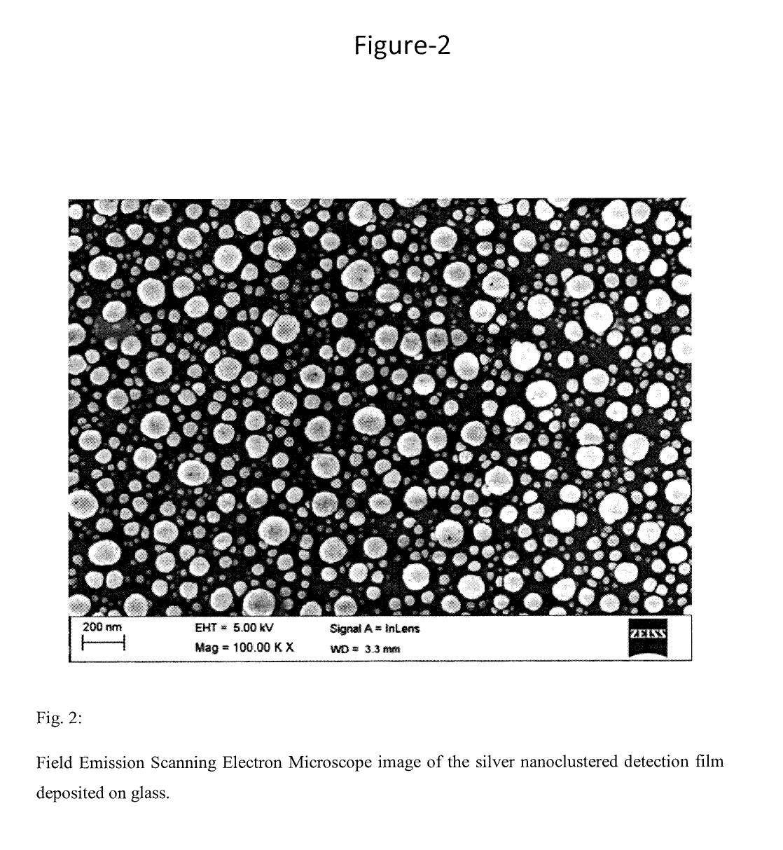

FIG. 2 shows FESEM image of silver nanocluster substrate

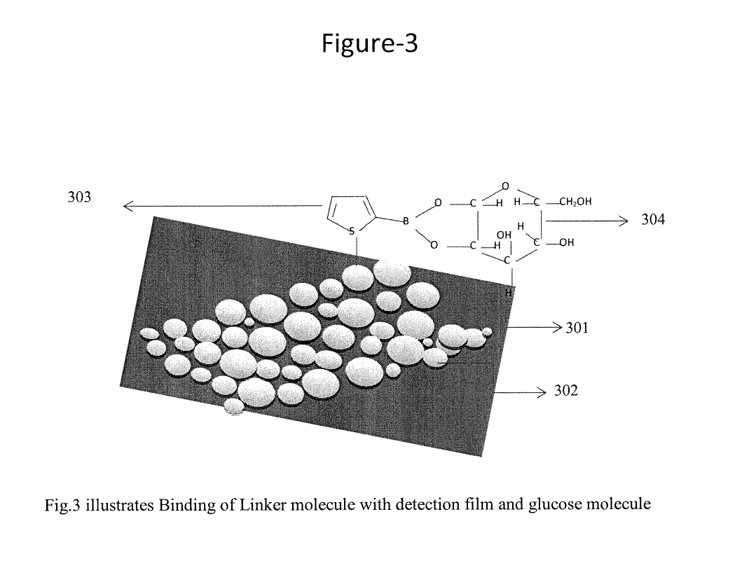

FIG. 3 illustrates the binding of linker molecule 303 with the detection film 302 and glucose molecule 304

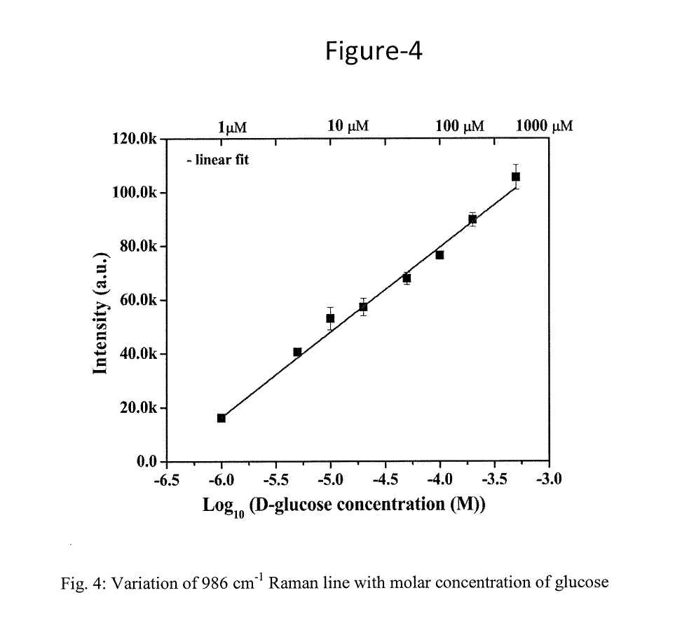

FIG. 4 illustrates an example showing the correlation between SERS signal intensity and concentration of glucose.

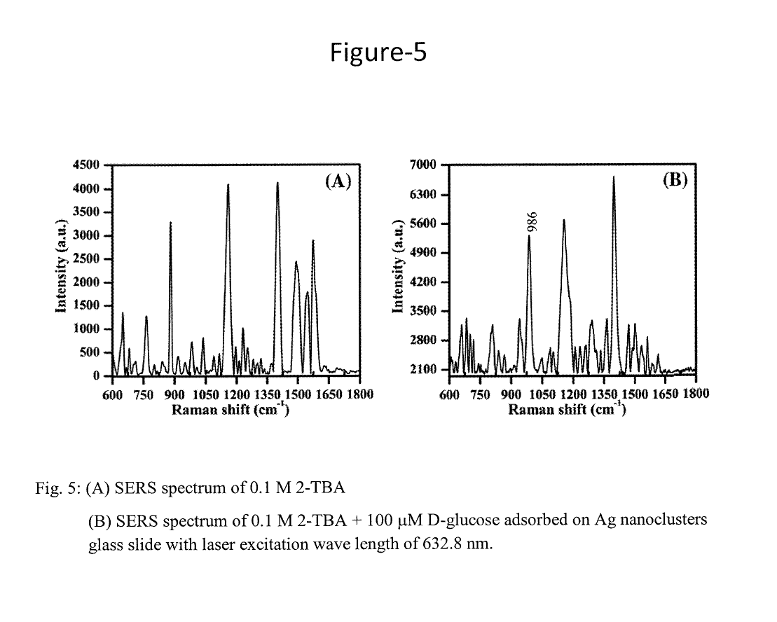

FIG. 5 illustrates (A) SERS spectrum of 0.1 M 2-TBA and (B) SERS spectrum of 0.1 M 2-TBA+100 μM D-glucose adsorbed on Ag nanoclusters glass slide with laser excitation wave length of 632.8 nm.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION OF THE INVENTION

The following description with reference to the accompanying flowchart is provided to assist in a comprehensive understanding of exemplary embodiments of the invention as defined by the claims and their equivalents. It includes various specific details to assist in that understanding but these are to be regarded as merely exemplary.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

It is to be understood that the singular forms “a,” “an,” and “the” include plural referents unless the context clearly dictates otherwise. Features that are described and/or illustrated with respect to one embodiment may be used in the same way or in a similar way in one or more other embodiments and/or in combination with or instead of the features of the other embodiments.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof

In the present invention, the term body fluid includes saliva or urine. Also, the notation “body fluid” means “body fluid or body fluid sample”. Likewise the notation “urine (sample)” means “Urine or Urine sample” and “Saliva (sample)” means “Saliva or Saliva sample”.

In the context of the present invention, an Analyte is a substance or chemical constituent that is of interest in an analytical procedure.

The present invention will be more clearly described with reference to the flowchart showing embodiments thereof.

According to an embodiment of the present invention, FIG. 1 is a flowchart illustrating the method 100 to determine the micro molar concentration of glucose in a body fluid. A detection film 101 is fabricated on a glass substrate using Physical Vapor Deposition Technique. Further, preparing 102 an analyte solution having body fluid whose glucose concentration is to be determined and linker molecule in pre-determined concentration. Now dropping 103 the solution on the detection film. Then permitting 103 the surface to dry under ambient condition. Finally recording and analyzing 105 the strong SERS signal in the Raman Spectra and determining 106 the micro molar concentration of glucose by measuring strong SERS signal.

According to an embodiment of the present invention, a detection film 302 needs to be prepared on which the body fluid (sample) can be dropped to carry out the enhancement process. A detection film 302 is a uniform layer of silver nanocluster which is deposited on the glass substrate. Silver nanoclusters are prepared using physical vapor deposition method. The detection film 302 is prepared by depositing the Silver nanocluster on the glass surface using the inert gas phase condensation technique. Silver Nano particles are sputtered on the glass surface and then it is allowed to go through an aggregation zone in the presence of an inert gas, where the Silver Nano particles are agglomerated into clusters and then deposited on the glass surface. The glass surface is sonicated and cleaned using iso-propan-2-ol, acetone and Milli-Q water before the deposition of the Silver nanocluster film.

The advantage of the producing detection film 302 through this production method is that it has no other chemical residues that are present in chemically prepared nanoclusters. Hence the produced detection film 302 is completely free from chemical contamination and also contributes to a good signal intensity that scales with glucose concentration.

Linker molecule is a molecule which should have selective adsorption for glucose molecule present in the body fluid as well as it should have strong affinity for detection film. The linker molecule used in this invention is a boronic acid derivative. More specifically, 2-thienylboronic acid has been used as a linker molecule. The proposed 2-Thienylboronic acid in this invention, act as a linker molecule that attaches directly to both the Silver nanocluster film surface and to the glucose molecule without any other intermediate molecule for functionalization. 2-thienylboronic acid gives strong SERS signal when being radiated.

In order to determine the glucose concentration level, an analyte solution comprising linker molecules and the body fluid is provided onto the Silver nanocluster film substrate surface. The linker molecules have unique binding properties which helps them to selectively bind to glucose molecule present in the analyte solution.

Now, when this analyte solution is being provided on the detection film 302 present on the glass surface, the linker molecules will bind to both, the detection film 302 as well as glucose molecules present in the Analyte solution. The detection film is then allowed to dry under ambient condition. The SERS substrate is then analyzed using Surface Enhanced Raman Spectroscopy. A new Raman line is observed in the SERS spectrum that did not exist in glucose or the substrate used. The method was therefore very effective for detecting the glucose.

The measuring of the SERS signal is thus directly related to the number of glucose molecules in the Analyte solution (sample). During the study it is observed that, the linker molecules gave strong SERS signal new characteristic line at 986 cm⁻¹ when being radiated. In this way, through the present method the detection of glucose concentration in body fluid is more sensitive.

It is an advantage of the present invention that there is no requirement of needle insertion through the skin, surgery or body penetrations, so the patient will not hurt during the method of glucose determination.

It is an advantage of certain embodiments of the present invention that an improved technique is provided for (directly) determining the concentration of the glucose in a body fluid, which is more sensitive technique than techniques known in the art.

Other aspects, advantages, and salient features of the invention will become apparent to those skilled in the art from the following detailed description, which, taken in conjunction with the annexed drawings, discloses exemplary embodiments of the invention.

### EXPERIMENTAL RESULTS

Fabrication of Detection Film on Glass Substrate:

During the experiment, silver nanocluster detection films were deposited on 1 cm² glass slides using inert gas phase condensation technique. A nanocluster deposition system was used for deposition of silver nanoclusters on the glass substrate. The substrate glass slides were sonicated and cleaned using iso-propan-2-ol, acetone and Milli-Q water before deposition of clusters. Surface morphological studies of silver nanocluster films were carried out using Field Emission Scanning Electron Microscopy (FESEM). FIG. 2 displays the FESEM image of the silver nanocluster detection film deposited on glass substrate.

Preparation of Analyte Solution having Body Fluid and Linker Molecule

A 0.1M stock solution of linker molecule i.e. 2-thienylboronic acid (2-TBA) was prepared and stored. A sample body fluid having different molar concentration (500 μM to 5 μM) of glucose was prepared by sequential dilution method. Analyte solutions were prepared by mixing 500 μL of the 0.1 M linker molecule solution and 250 μL solution of body fluid solution of different molar concentration.

Monitoring the Detection Film with Linker Molecule:

A 50 μL volume of the analyte solution was dropped on the silver nanocluster detection film 302 deposited on glass substrate 301 and allowed to dry naturally overnight under ambient condition. The linker molecule present in the analyte solution allows itself to attach with silver nanocluster detection film through the sulphur atom of the thienyl group of linker molecule 303 as well as the glucose molecule 304 to the OH—B—OH part of the linker molecule 303 as depicted in FIG. 3.

Recording and Analyzing the SERS Signal in the Raman Spectra:

SERS measurements were carried out on these SERS substrates using a micro-Raman Spectrometer. The experimental method used for Raman scattering consists of laser beam of wavelength 632.8 nm that is made to scatter from the SERS substrate and the intensity of the scattered beam is monitored as a function of wavelength. An intense peak at 986 cm⁻¹ Raman line appeared in the SERS spectrum that did not exist in glucose or the substrate used. The intensity of this new characteristic line at 986 cm⁻¹ was measured in the Raman spectrum as a function of glucose concentration. During the study, it was observed to be correlated linearly with the glucose molar concentration in the range 5 to 500 micromoles. FIG. 4 shows correlation of the intensity of this peak with the molar glucose concentration of the Analyte solution.

As such, the present method is promising as it is a direct detection method in which D-glucose molecule is attaching and influencing the properties of the linker molecule directly. In addition a new and very strong Raman signal is seen in the Raman data which enhances the Raman signal intensity and hence helpful in determining the micro molar concentration of glucose in body fluid.

We claim:

1. A method of determining micro-molar concentration of glucose in a body fluid using Surface Enhanced Raman Spectroscopy, the method comprising:
a. preparing an analyte solution by mixing body fluid whose glucose concentration has to be determined and pre-determined concentration of linker molecule solution capable of selectively attaching to the glucose molecule,
b. dropping the said solution on surface composed of
i. a glass substrate;
ii. a detection film deposited on the surface of the glass substrate capable of binding the linker molecule;
c. permitting the said surface to dry naturally under ambient conditions;
d. recording and analyzing the glucose specific strong SERS signal in the Raman Spectra; and
e. determining the presence of glucose at micro-molar level by measuring the strong SERS signal.

2. The method of claim 1, wherein the linker molecule is a boronic acid derivative, more specifically, 2-thienylboronic acid.

3. The method of claim 1, wherein the body fluid includes saliva or urine.

4. The method of claim 1, wherein linker molecule solution and the body fluid are mixed in the ratio 2:1.

5. The method of claim 1, wherein the detection film is a silver nanocluster film prepared by physical vapor deposition technique and does not have other chemical residue.

6. The method of claim 1, wherein the detection film is deposited on glass substrate using the inert gas phase condensation technique.

7. The method of claim 1, wherein the said linker molecule specifically attaches to the silver nanocluster film through the sulphur atom and D-glucose molecules through OH—B—OH part respectively.

8. The method of claim 1, wherein the strong Raman Signal with intensity peak of the new characteristic Raman line ranges from 800 cm⁻¹ to 1000 cm⁻¹.

9. The method according to claim 1, wherein determining micro molar concentration level of glucose in body fluid includes correlating the SERS signal with the amount of linker molecules.

10. A method of determining micro-molar concentration of glucose in a body fluid using Surface Enhanced Raman Spectroscopy, which is direct and non-invasive.
